(19) United States
(12) Patent Application Publication
(10) Pub. No.: US 2003/0031350 A1
(43) Pub. Date: Feb. 13, 2003

(54) Methods for large scale protein matching

(75) Inventor: Benjamin R. Halpern, San Jose, CA (US)

Correspondence Address: CELERA GENOMICS CORP., ATTN: WAYNE MONTGOMERY, VICE PRES, INTEL PROPERTY, 45 WEST GUDE DRIVE, C2-4#20, ROCKVILLE, MD, 20850, US

(73) Assignee: PE CORPORATION (NY), Norwalk, CT

(21) Appl. No.: 10/087,541

(22) Filed: Mar. 1, 2002

Related U.S. Application Data
(60) Provisional application No. 60/274,254, filed on Mar. 9, 2001.

(51) Int. Cl. C12P 21/06; G06K 9/00

(52) U.S. Cl. 382/129; 435/69.1

(57) ABSTRACT

The present invention provides methods for matching a sample of an unknown query peptide to a database of known peptides. The methods described herein allow for the rapid, sensitive, and selective identification of an unknown query peptide, which enables the development of high throughput protein identification. The methods described herein also allow for mass spectrometry data for a query peptide to be categorized and weighted according to its quality. Furthermore, the methods described herein provide robust identification of modified query proteins by either anticipating modifications or adjusting for modified peptide masses.

### BACKGROUND OF THE INVENTION

1. Field of the Invention

The present invention relates to the field of proteomic analysis, and is especially related to providing methods for matching proteins analyzed by mass spectrometry to known amino acid sequences in a database.

2. Description of Related Art

Tandem mass spectrometry (“MS/MS”) techniques have been proven for analyzing peptides. In tandem mass spectrometry, the peptide is applied to a first mass spectrometer which serves to select, from a mixture of peptides, a target peptide of a particular mass or molecular weight. The target peptide is then activated or fragmented to produce a mixture comprising the intact peptide and various component fragments, typically peptides of smaller mass. This mixture is then applied to a second mass spectrometer which generates a fragment spectrum. This fragment spectrum will typically be expressed in the form of a bar graph having a plurality of peaks, each peak indicating the mass/charge ratio of a detected fragment.

The fragment spectrum can then be used to identify the target peptide. Previous approaches have typically involved using the fragment spectrum as a basis for hypothesizing one or more candidate amino acid sequences. This procedure has typically involved human analysis by a skilled researcher, although at least one automated procedure has been described John Yates, III, et al, Techniques In Protein Chemistry II (1991), pp. 477-485, incorporated herein by reference. The candidate sequences can then be compared with known amino acid sequences of various proteins in the protein sequence libraries.

Genome sequencing efforts have yielded a vast amount of raw DNA sequence information, which in turn has yielded a vast amount of protein sequence information. As the amount of protein sequence information increases, so does the amount of information related to their implied digest and fragmentation products.

Two circumstances have combined to make speed an important consideration in the identification of peptides through database searching with mass spectrometry fragmentation spectra.

The first circumstance is that the database of known peptides is growing rapidly. One cause of this growth in known peptides is the growth in the number of known proteins being catalogued in databases; this results in the number of their implied digest products correspondingly increasing. A second cause is that the human genome has been sequenced and many other genomes are being sequenced; these genomes likewise imply large numbers of peptides through their theoretical translation and digestion.

The second circumstance is that there are more fragmentation spectra being produced from unknown peptides. In this sort of situation, capability or capacity itself leads in turn to increased demand. The several new techniques for the automated collection of fragmentation spectra have led to the popularity of high throughput experiments with peptides.

The new techniques for the automated collection of fragmentation spectra include the capability of new MS machines for the automated selection of candidate peptides for fragmentation from the continuous input from an LC column. Another new technique is the ICAT protocol for collecting thousands of peptides from expressed genes. By combining these two techniques, approximately a thousand fragmentation spectra can be produced within a three hour run of the machine. The MALDI technique also lends itself to high throughput.

Interpretation of the fragment spectra so as to produce candidate amino acid sequences is time-consuming, often inaccurate, highly technical and in general can be performed only by a few laboratories with extensive experience in tandem mass spectrometry. Reliance on human interpretation often means that analysis is relatively slow and lacks objectivity. Approaches based on peptide mass mapping are limited to peptide masses derived from an intact homogenous protein generated by specific and known proteolytic cleavage and thus are not generally applicable to mixtures of proteins.

One impediment to high throughput protein identification by mass spectroscopy is the presence of modifications on proteins that effect their mass, leading to wasted query mass ratios and unintended hits. Methods in the prior art for addressing this problem employ the complementary y-ion to a b-ion, and vice versa, because if the modification is in the ion, it isn't in its complement, and vice versa. One unfortunate side effect of this method is that by doubling the number of query mass ratios, the noise level is also doubled. See Clauser K R, et al, Proc Natl Acad Sci USA 92: 5072-6 (1995).

There is a need for increased speed and flexibility in peptide identification, leading to increased sensitivity and selectivity, which can facilitate high-throughput peptide identification projects. These projects in turn may lead to new beneficial drug discoveries, better understanding of biological processes, and consequentially better products and methods for maintaining health and benefiting agriculture.

Furthermore, there is a need for increased sensitivity and selectivity in high-throughput identification of peptides.

Furthermore, there is a need to minimize the effect of peptide modifications on high-throughput identification of peptides.

Furthermore, when the mass of a modification is known, there is a need to employ this mass information to enhance the robustness of identification of a modified query peptide.

Finally, there is a need for enhanced speed as well as robustness when identifying query proteins containing the most common types of modifications.

### BRIEF SUMMARY OF THE INVENTION

A detailed description of each of these elements and the operation of the method is provided below. All references cited herein are incorporated by reference in their entirety.

In one aspect, the invention relates to a method for comparing a query peptide to a plurality of database peptides using mass spectrometry data from the query peptide and a pre-calculated peptide index.

In another aspect, the invention relates to a method for increasing sensitivity and selectivity in the identification of peptides from their mass spectrometry fragmentation spectra by identifying the various categories of hits and optimizing a set of weights assigned to these categories.

In another aspect, the invention relates to a method for minimizing the deleterious effect of a modification of a query peptide when comparing the modified query peptide to a plurality of database peptides.

In another aspect, the invention relates to a method for employing the mass information of a known modification of a query peptide to enhance the robustness of its identification.

In another aspect, the invention relates to a method for increasing the speed of identifying a modified query peptide by comparing the modified query peptide to a plurality of database peptides augmented by a plurality of modified database peptides.

### BRIEF DESCRIPTION OF THE DRAWINGS

FIG. 1 presents a flowchart illustrating the preparation of an index table in one embodiment of the invention.

FIG. 2 presents a flowchart illustrating the searching of an index table in one embodiment of the invention.

### DETAILED DESCRIPTION OF INVENTION

Definitions

For the purposes of this invention, “peptide” refers to a sequence of amino acids. A “peptide database” refers to a list of peptides. A “peptide index” refers to identification information for locating a specific peptide in a peptide database. In one embodiment, a peptide index refers to an offset value from the beginning of the database.

For the purposes of this invention, an “initial string” of a peptide refers to a subsequence of the peptide beginning at the peptide's first amino acid. Similarly, a “terminal string” of a peptide refers to a subsequence of the peptide ending at the peptide's last amino acid. Both the initial string and terminal string may refer to the entire peptide.

For the purposes of this invention, when a peptide is fragmented and the charge is retained on the N-terminal cleavage fragment, the resulting ion is labelled as a “b-ion”. Similarly, if the charge is retained on the C-terminal cleavage fragment, it is labelled a “y-ion”. Masses for b-ions are calculated by summing the amino acid masses and adding the mass of a proton. Masses for y-ions are calculated by summing, from the C-terminal, the masses of the amino acids and adding the mass of water and a proton.

For the purposes of this invention, the mass of a peptide is the sum of the masses of its constituent amino acids. The set of “initial masses” of a peptide consists of the masses of all of its possible initial strings. Similarly the set of “terminal masses” of a peptide consists of the masses of all its possible terminal strings. The set of “associated masses” of a peptide consists of the union of the set of initial masses and the set of terminal masses. Except as otherwise noted, the terms “mass,” “mass ratio” and “mass/charge ratio” are used interchangeably for the purposes of this invention.

The set of “predicted mass ratios” of a peptide is the set of mass/charge values expected to result from performing a mass spectrometry measurement on a sample of the peptide.

For the purposes of this invention, the “index table” refers to a data structure whose records are indexed by discrete mass values and whose fields contain references to the associated peptides responsible for those values. The “allowed values” of an index table refers to the range of allowable values for the table's index. The “row” of an index table refers to a record, and a “column” refers to a field.

For the purposes of this invention, the “query peptide” refers to a peptide to be compared against a peptide database. A “query spectrum” is a mass spectrometry fragmentation spectrum of a sample of the query peptide comprising a plurality of mass/charge values. For the purposes of this invention, a query spectrum does not include any intensity values from the mass spectrometry data. The set of “query masses” and “query mass ratios” refers to a set of masses derived from the query spectrum. The subset of “primary query masses” and “primary query mass ratios” are those derived directly from peaks in the fragmentation spectrum. The subset of “complementary query masses” and “complementary query mass ratios” are those calculated by subtracting the primary query masses from the mass of the full query peptide.

For the purposes of this invention, a “hit” represents a peptide index located at a mass value of the index table, wherein the absolute difference between mass value and the a query mass is smaller than a predefined tolerance value.

For the purposes of this invention, a “peak mass ratio” is a query mass ratio derived by adjusting a measured mass/charge ratio for its putative isotope patterns and/or charge.

For the purposes of this invention, a “modification” is a change in the mass ratio of a peptide, either by one of its amino acids being changed, or by its N-terminal or C-terminal group being changed. An amino acid may be modified by being phosphorylated, glycosylated, or replaced with a different amino acid. The “location” of a modification is the location of the modified amino acid. For the purposes of this invention, the “spectral range” of a peptide ranges from zero to the molecular weight of the unmodified peptide.

For the purposes of this invention, the “difference mass” of a modified query peptide refers to the difference between the molecular weight of the modified query peptide and the molecular weight of the unmodified query peptide. For example, if the modification were a phosphorylation, the difference mass would be the mass of the phosphoryl group. The “modification mass ratio” refers to the mass/charge ratio of the first modified b-ion of a modified peptide.

Basic Search Method Using a Pre-calculated Index Table

The search methods of this invention require the pre-calculation of an index table. The index table is indexed by mass in discrete increments within a range of allowed values. For example, an index table could contain the values from 0.01 to 30,000 Daltons, in increments of 0.01 Dalton, resulting in a 3,000,000-row table.

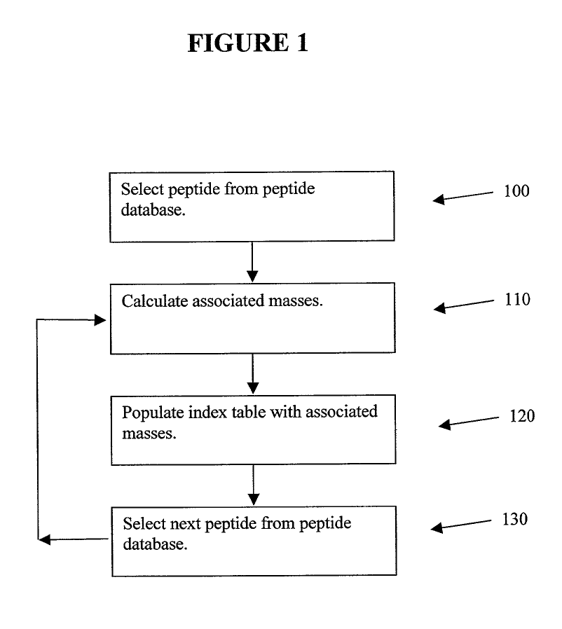

Referring to FIG. 1, generation of the index table involves selecting a peptide from the peptide database (Step 100), calculating the set of associated masses for the peptide (Step 110) and for each associated mass, placing a peptide index into the row in the index table corresponding to that mass (Step 120). Steps 100-120 are then repeated for each peptide in the peptide database (Step 130).

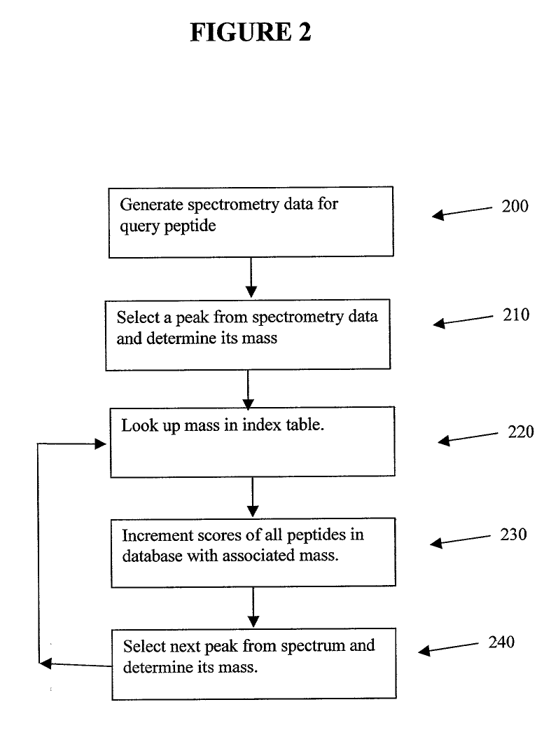

Referring to FIG. 2, a search involves comparing the set of query masses against the set of all associated masses for all peptides in the peptide database. In one embodiment, a search involves generating mass spectrometry data from the query peptide (Step 200), identifying a peak from the spectrum and determining its mass (Step 210), looking up the entry in the index table corresponding to that mass (Step 220), and incrementing the scores of all peptides in the database having the same associated mass (Step 230). Steps 200-230 are then repeated for every peak in the spectrum (Step 240). Finally, those peptides with the greatest number of hits are identified.

It is possible to create an index table that is both efficient with respect to both memory and speed. In one embodiment, the index table is calculated in two passes. In the first pass, the number of entries for each row is calculated. Based on the number of entries in each row, the proper amount of memory for that row is allocated. In the second pass, the rows are populated with peptide indices referencing the peptides responsible for the associated masses corresponding to each row.

In one embodiment, a search is performed as follows: A score value is allocated and initialized for each peptide in the peptide database. For each query mass, the corresponding row in the index table is referenced, all of the peptide indices in the row are looked up, and all score values associated with those peptide indices are incremented.

A further embodiment employs a tolerance value for matching a query mass to a mass associated to a peptide in the peptide database. A query mass can hit an initial mass if the difference between the query mass and the expected N-terminal mass of the associated initial string is within a tolerance of the initial mass. Similarly, a query mass can hit an terminal mass if the difference between the query mass and the expected C-terminal mass of the associated terminal string is within a tolerance of the terminal mass. In this embodiment, a search is performed as follows: As in the previous example, a score value is allocated and initialized for each peptide in the peptide database. However in addition to referencing the row corresponding to the query mass, all neighboring rows within the specified tolerance are also referenced. In a manner similar to the previous example, all of the peptide indices in all of the referenced rows are looked up, and all score values associated with those peptide indices are incremented.

Weighted Search Method: Categories of Hits

In one embodiment, the search method employs a set of weighting factors to the various categories of peaks in the query spectrum, as experimental data indicate that some categories of peaks may yield more predictive hits than others. Peaks in the query spectrum may be categorized by several criteria. One such criterion is the type of ion which produced the peak, such as a y-ion, b-ion, a-ion, or immonium ion. Another criterion is whether the peak is a primary or complementary peak.

In mass spectrometry, a sample of a peptide is fragmented into a plurality of subfragment ions, and the mass/charge ratios of these ions are determined. Categories of subfragment ions are well known in the art, including y-ions, b-ions, a-ions, and immonium ions. For example, it has been observed that y-ions are about twice as common as b-ions in some common settings in common machines. Thus, the number of hits involving predicted y-ions should be more predictive than the number of hits involving predicted b-ions. Consequently, if the hits from those more predictive categories are weighted more heavily the ensuing query peptide identification may be more likely to be true.

In this embodiment, a set of ion types is selected. In a preferred embodiment, the set of singly-charged y-ions and b-ions is selected. Then the set of all possible subfragment ions is calculated for each peptide in the peptide database, the predicted mass/charge ratio is calculated for each subfragment ion, and the peptide index is populated according to the set of predicted mass/charge ratios as described in the section above.

In this embodiment, the query spectrum is examined for peaks corresponding to ions of the selected set of ion types. The set of query mass ratios is determined by selecting those peaks believed to correspond to the selected set of ion types.

Sometimes the mass ratio of the peak itself is a query mass ratio, as when the isotope pattern that this peak belongs to suggests that it has a single charge. When the isotope pattern suggests that the ion giving rise to the peak has a charge of 2, then its mass ratio multiplied by 2, minus the mass of hydrogen, may be used as a query mass ratio. Similarly, when the isotope pattern suggests other charges, the mass ratio of the peak is adjusted to the equivalent singly charged, mono-isotopic mass ratio before it is used as a query mass ratio.

The set of query mass ratios can be divided into primary and complementary query mass ratios. Those derived directly from the query spectrum are referred to as the set of primary query mass ratios. In one embodiment, a complementary query mass ratio C is calculated according to the following formula:

C=Q+2H−P

where Q is the molecular weight of query peptide, H is the mass of hydrogen, and P is the primary query mass ratio. The set of query mass ratios comprises the union of the sets of primary and complementary mass ratios.

Determining an Optimal Set of Weights

Because the quality of data in a fragmentation spectrum can vary from peak to peak, searching a peptide database with data derived from a fragmentation spectrum often fails to produce matches with sufficient specificity and sensitivity. In one embodiment, this invention categorizes peaks from the fragmentation spectrum according to their perceived quality and assigns higher weights to higher quality peaks. For example, the quality of a peak can vary according to whether the peak represents a y-ion or a b-ion; specifically, since y-ions tend to be twice as prevalent as b-ions in common machines at common settings, it follows that the number of hits involving y-ions should be roughly twice as predictive as those of b-ions. In another example, the quality of a peak can also vary proportionally to its intensity.

In one embodiment, the weights that are assigned to each category of peak are calculated through the use of learning examples. A learning example comprises a query spectrum for which the correct peptide is known. The weights assigned to the categories are adjusted and tuned on the learning examples so that the known answer among the database peptides stands out from the crowd of possibilities most sharply.

In an illustrative example, suppose there are n peptides in the peptide database, that there are m categories of hits, that Hᵢⱼ is the number of hits in category j for peptide i, and that Wⱼ is the weighting value for category j. In this example, Xₗ is the score for peptide i and is calculated as follows:

$${X}_{i} = {\sum }_{j} {W}_{j} * {H}_{i , j}$$

The average score, \overline{X}, is calculated as follows:

$$\overline{X} = \frac{1}{n} {\sum }_{i} {X}_{i}$$

The population variance, σ², for \overline{X} is calculated as follows:

$${σ}^{2} = ( \frac{1}{n} ) {\sum }_{i} {( {X}_{i} - \overline{X} )}^{2}$$

In a learning sample, the query peptide is known and is present in the peptide database at position q. Let X_{q} be the score calculated for the query peptide. Define the normal deviate, D, as follows:

$$D = \frac{{X}_{q} - \overline{X}}{σ}$$

A desirable set of weights is one that distinguishes the score for the correct match, in this case X_{q}, from all other scores. In this example, therefore, it is desirable to set the weights to maximize D.

In one method for determining optimal weights, a covariance value C_{ab} is used. The value C_{ab} represents the covariance between categories a and b, and is calculated as follows:

$${C}_{a b} = ( \frac{1}{n} ) {\sum }_{i} ( {H}_{i a} - \overline{X} ) ( {H}_{i b} - \overline{X} )$$

It follows that the variance calculation described above can also be expressed in terms of the weights and the covariance:

$${σ}^{2} = {\sum }_{a = 1}^{m} {\sum }_{b = 1}^{m} {W}_{a} {W}_{b} {C}_{a b}$$

Taking the derivative with respect to a specific weight value Wₖ yields:

$$\frac{\partial {σ}^{2}}{\partial {W}_{k}} = 2 {\sum }_{b = 1}^{m} {W}_{b} {C}_{b k}$$

Similarly, the partial derivative of N² with respect to a specific weight value Wₖ can be expressed as:

$$\frac{\partial {N}^{2}}{\partial {W}_{k}} = \frac{{σ}^{2} 2 ( {X}_{q} - \overline{X} ) ( {H}_{q k} - {\overline{X}}_{k} ) - {( {X}_{q} - \overline{X} )}^{2} 2 {\sum }_{a = 1}^{m} {W}_{a} {C}_{a k}}{{( {σ}^{2} )}^{2}}$$

Setting this to zero, and simplifying by assuming that X_{q}≠\overline{X}, we get:

$${σ}^{2} ( {H}_{q k} - {\overline{X}}_{k} ) = ( {X}_{q} - \overline{X} ) {\sum }_{a = 1}^{m} {W}_{a} {C}_{a k}$$

$${\sum }_{a = 1}^{m} {W}_{a} {C}_{a k} = \frac{{σ}^{2} ( {H}_{q k} - {\overline{X}}_{k} )}{( {X}_{q} - \overline{X} )}$$

Using vector and matrix notation, and defining the vector d such that:

dₐ=H_{qa}−\overline{X}ₐ

Then:

$$W C = ( \frac{{σ}^{2}}{{X}_{q} - \overline{X}} ) d$$

$$W = ( \frac{{σ}^{2}}{{X}_{q} - \overline{X}} ) d {C}^{- 1}$$

This equation can be solved to yield an optimal set of weights for the learning example q.

The invention uses a set of learning examples to determine a set of weights to use for subsequent unknown peptides. For each learning example, a set of optimal weights is calculated and normalized so the sum of their squares is 1. Then the average over the set of learning examples of each of these normalized weights is used in searches with new unknown peptides. A desirable set of weights are those which maximize the normal deviate.

Once a set of weights is determined, the weights are employed in assaying unknown query spectra, having the reasonable hope that they improve identification of an unknown query peptide. In one embodiment, separate index tables are created for predicted mass ratios of different ion types. In an alternate embodiment, separate index tables are created for primary and complementary mass ratios. In these embodiments, each index table has a weight associated with it. During the search, score values are incremented. The score value for each index table is then multiplied by its weight. Finally, the score values for each peptide in the peptide database are summed across index tables.

In a further embodiment, separate index tables are created for separate, orthogonal criteria. For example, separate index tables can be created according to whether the query mass ratio represents a b-ion or a y-ion, and whether query mass ratio represents a peak mass ratio or a complement mass ratio. In this example, four separate index tables are created: one for b-ions, one for y-ions, one for peak mass ratios, and one for complement mass ratios. Comparing a query peptide to these tables results in four separate counts. Each count is then multiplied by the table's corresponding weight, and all weighted counts are summed to produced a weighted score for the query protein.

Minimizing the Effect of Peptide Modifications

Many peptides contain modifications such as post-translational modifications, including phosporylation and glycosylation. Other modifications include substitution of amino acids and changes in the N-terminal or C-terminal group. Such modifications change the peptide's mass, making it difficult for that peptide to be identified through mass spectrometry. Specifically, such modifications result in some of the ions of the query peptide being chemically different from the corresponding ions of the unmodified peptide. Hence some of the query mass ratios will not match their predicted mass ratios. When the location of the modification is unknown, then it is also unknown which ions and their measured mass/charge ratios have been effected by the modification. Experimental evidence indicates that when there is a modification of an unknown query peptide, about half of the query peptide's mass ratios are observed to not correspond to a predicted mass ratio for the correct peptide. That is, about half of the query masses of a modified query peptide are not expected to distinguish the correct peptide from the other peptides. These modified query masses are not only wasted, in that they do not contribute to the score of the correct database peptide, but are actually harmful, in that they increase the scores of incorrect database peptides. In one embodiment, this invention identifies modified query masses.

The difference between the molecular weight of the modified query peptide and that of the unmodified query peptide is called the “difference mass.” If the difference mass is not known, then the modified mass ratios in the query spectrum should be excluded from comparison. In the case where the difference mass is known, that information should be used to adjust the query mass ratios, thus increasing the selectivity and sensitivity of the search. In one embodiment, the query mass ratios are adjusted by subtracting the difference mass from them.

In one embodiment, the search method identifies the modified query masses of a modified query protein by dividing the spectral range of the query peptide into intervals and performing separate searches for each interval. In a further embodiment, these modified query masses are excluded from comparison with the peptide index. In an alternate embodiment, these modified query masses are adjusted before being used for comparison with the peptide index.

The range from zero to the unmodified query peptide's mass is called the spectral range. Given the mass of a query peptide, all query mass ratios higher than the predicted mass can be ascribed to modification. In one embodiment, the spectral range is divided into intervals, and separate searches are performed over each interval.

In one embodiment, the query peptide's spectral range is divided into m equal intervals. Consider one such interval from mass j to mass k, and assume that the modification mass ratio lies in the [j,k] interval. By assuming that the modification lies in the [j,k] interval, a set of modified query mass ratios can be identified. These identified mass ratios can then be dropped from comparison if the difference mass is unknown, or adjusted if the difference mass is known. Different sets of mass ratios can be identified, for example one set can be identified by comparing to predicted b-ion mass ratios, and another set can be identified by comparing to predicted y-ion mass ratios. Specifically, all the query mass ratios greater than k are dropped or adjusted when looking for hits against predicted b-ion mass ratios; all the query mass ratios greater than molecular weight (2H−j) are dropped or adjusted when looking for hits against predicted y-ion mass ratios

In this embodiment, after the query peptide's spectral range is divided into m intervals, a separate search is performed on each interval with each search assuming that the query peptide's modification lies in that search's interval. After performing the separate searches, the scores from each search are summed up, and the peptide with the highest score over all of the searches is determined to be the best match to the query peptide.

The method of this embodiment increases the sensitivity and specificity of a modified query protein search by altering the distribution of hits in the search process. To understand the expected advantage of identifying modified query mass ratios in the search process, it is first neccessary to examine the expected distribution of hits in a normal search where one interval covers the whole modified query peptide.

Suppose a query peptide is compared to a peptide database consisting of k peptides. A histogram F can be constructed wherein F_{b} represents the number of database peptides receiving b hits. The fraction of peptides in the database receiving b hits, D_{b}, can be calculated thus:

$${D}_{b} = \frac{{F}_{b}}{k}$$

If the search is defined as a number of trials wherein each query mass represents a trial, and if success is defined as the query mass hitting a peptide in the peptide index, then D (and F) can be seen to follow a binomial distribution. The variance of a binomial distribution is proportional to the number of trials; specifically the variance of the binomial distribution (n,p), where n is the number of trials and p is the probability of success per trial, is np(1−p). In other words, the variance of D (and F) is proportional to the number of query mass ratios used in the search. A desirable probability density of D (and F) represents a small number of sequences receiving a high number of hits, providing a sharp contrast between a true hit and noise. The binomial distribution approaches this ideal for lower values of n, especially for small values of p. Limiting a search to a short interval reduces the number of query mass ratios, or n, which in turn leads to a more useful probability density function for D (and F).

In an illustrative example, two searches are performed and the results are used to calculate the histogram vectors H1 and H2. In this example, assuming that H1 and H2 are uncorrelated, it follows that H1 and H2 are random variables with the same density functions as F and D, above. Now assume that the first search consists of n query masses and the second search consists of 2n query masses. It follows that the variance of the H2 is twice that of H1. Therefore, because searching over a smaller interval reduces the number of query masses, interval searches have a smaller variance than searches over the entire peptide.

For larger peptide databases, that is, for increasing values of k, the difference becomes even more pronounced. Although the underlying density, D, remains constant, the raw values in the histogram F increases proportionally to k, resulting in a closer approximation to the desired binomial distribution. By dividing the peptide into m intervals and performing m searches, the size of the peptide database is effectively increased by a factor of m. Thus, the method described herein performs the dual purpose of designed a desirable probability density function for the results, as well as making the results correlate more closely to the desired function. However, an expected disadvantage to performing m searches and effectively increasing the number of peptides in the peptide database by a factor of m is that this approach also increases F by a factor of m, raising the tail of the distribution and slowing its dropoff.

When the number of intervals is small, one doesn't drop as many modified query masses as when the number of intervals is larger. But as one does more searches, the disadvantage described above increases. Experimental evidence indicates that 6 is about the optimal number of intervals to use. The location in the tail of the number of hits on the correct peptide, and the manner of decay of the tail have been estimated. Experimental evidence indicates that for m˜6, the expected advantage of eliminating modified query masses outweighs the expected disadvantages by a factor of 30. Experimental evidence further indicates that for m˜6, the expected advantage of adjusting modified query masses outweighs the expected disadvantages by a factor of 5000.

In one embodiment, the number of query masses in an interval is further reduced by identifying and eliminating modified query masses. For example, as illustrated above, if half of the query masses are eliminated, the variance of the resulting distribution is halved.

In an alternate embodiment, the modified query masses are identified and then adjusted. In a further embodiment, the modified query masses are adjusted by subtracting the known difference mass. Although the adjusted modified query masses are not eliminated from comparison, their hits to peptide database are more likely to be correct than if left unadjusted. The method of this embodiment can be seen as a way to double the number of correct hits for a modified query protein.

Although the examples herein describe analysis of a singly-modified protein, one of ordinary skill in the art can readily comprehend how the described methods can easily be extended to analyze proteins containing two or more modifications.

Adding Modifed Peptides to the Peptide Database

In one embodiment, this invention provides a method for increasing the likelihood that an unknown modified query peptide will be correctly identified by adding appropriately modified peptides to the peptide database before proceeding with the construction of the index table.

It is well established in the art that the most common modifications to peptides apply only to certain amino acids. For example, only serine, threonine, and tyrosine are receptive to phosphorylation. Similarly, only cysteine and methionine are commonly oxidized. It is also well established in the art that some point mutations of amino acids are more common than others. For example, glutamate is often seen to be substituted for glutamine, and asparate for asparagine. Consequently, when a small set of common modifications is considered, the number of possible modifications of a given peptide in a peptide database is relatively small. For example, the average peptide with a molecular weight between 600 and 2,000 daltons has two phosphorylation sites. By this calculation, adding singly-phosphorylated peptide variants to a peptide database will increase its size by a factor of 3.

Experimental evidence indicates that three specific modifications account for the majority of modified peptides measured in tandem mass spectrometers: oxidation of methionine, mutation of glutamine to glutamate, and mutation of asparagine to aspartate. For one peptide database, it has been calculated that adding variant peptides incorporating these three classes of modification would increase the database's size by 40% to 150%. It is important to note that the size of the index table is mostly invariant relative to the size of the peptide database used to generate it, i.e. the larger peptide database does not result in a significantly larger index table. Nor is the speed of the search significantly affected by the more heavily populated index table. Therefore, a modest increase in the calculation time of the index table can result in improved sensitivity and selectivity of a search without having a noticable impact on searching speed.

Equivalents

The invention may be embodied in other specific forms without departing from the spirit or essential characteristics thereof. The foregoing embodiments are therefore to be considered in all respects illustrative, rather than limiting, of the invention described herein. Scope of the invention is thus indicated by the appended claims, rather than by the foregoing description, and all variants which fall within the meaning and range of equivalency of the claims are therefore intended to be embraced therein.

What is claimed is:

1. A method for comparing a query peptide to a plurality of database peptides comprising the steps of:
(a) constructing an index table, said index table comprising a plurality of records corresponding to a plurality of allowed mass values, said records comprising zero or more fields, said constructing step comprising the steps of:
(i) selecting a first peptide from said plurality of database peptides;
(ii) calculating a plurality of associated masses for said first peptide;
(iii) selecting a first associated mass from said plurality of associated masses;
(iv) referencing a first record from said plurality of records, said first record corresponding to said first associated mass;
(v) entering a first field into said first record, said first field comprising a first peptide index referencing said first peptide;
(vi) repeating steps (iii)-(v) for at least one other associated mass from said plurality of associated masses;
(vii) repeating steps (i)-(vi) for at least one other peptide from said plurality of database peptides; and
(b) generating a plurality of comparison scores, said plurality of comparison scores corresponding to said plurality of database peptides, said generating step comprising the steps of:
(i) generating a plurality of query mass values for said query peptide;
(ii) selecting a first query mass value from said plurality of query mass values;
(iii) referencing a second record from said plurality of records, said second record corresponding to said first query mass value;
(iv) selecting a second field from said second record, said second field comprising a second peptide index;
(v) selecting a first comparison score from said plurality of comparison scores, said first comparison score corresponding to said second peptide index;
(vi) incrementing said first comparison score;
(vii) repeating steps (ii)-(vi) for at least one other query mass value selected from said plurality of query mass values.

2. The method of claim 1 wherein said generating step (b)(i) comprises the step of performing mass spectroscopy on said query peptide.

3. The method of claim 2 wherein said mass spectroscopy is performed by a method selected from the set consisting of: Fourier transform ion cyclotron resonance (“FTICR”), quadrupole mass spectroscopy, ion trap mass spectroscopy, and time-of-flight mass spectroscopy.

4. The method of claim 1 wherein said calculating step (a)(ii) comprises the step of calculating a plurality of associated masses for said first peptide, said plurality of associate masses comprising a plurality of primary masses and a plurality of complementary masses.

5. The method of claim 1 wherein said generating step (b) further comprises the step of multiplying said first comparison score by a weight value, wherein said weight value is a function of the type of mass value.

6. The method of claim 5 wherein said type of mass value is selected from the group consisting of: y-ion, b-ion, peak mass, and complementary mass.

7. A method for comparing a query peptide to a plurality of database peptides comprising the steps of:
(a) constructing a first index table, said first index table comprising a first plurality of records corresponding to a plurality of allowed mass values, said records comprising zero or more fields; and
(b) constructing a second index table, said second index table comprising a second plurality of records corresponding to said plurality of allowed mass values, said records comprising zero or more fields; and
(c) calculating a plurality weight values, said weight values set according to the predictive value of said first and second index tables.

8. A method for comparing a modified query peptide to a plurality of database peptides comprising the steps of:
(a) generating a plurality of query mass values for said query peptide;
(b) identifying a set of query mass values from said plurality of query mass values, wherein said set corresponds to modified mass values;
(c) determining a spectral range for said query peptide;
(d) subdividing said spectral range into a plurality of equal intervals;
(e) performing a plurality of searches on said plurality of equal intervals.

9. The method of claim 8 further comprising the step of excluding said set of query mass values.

10. The method of claim 8 further comprising the step of adjusting said set of query mass values.

11. A method for comparing a query peptide to a plurality of database peptides comprising the step of constructing an index table, said index table comprising a plurality of records corresponding to a plurality of allowed mass values, said records comprising zero or more fields, said constructing step comprising the steps of:
(i) selecting a first peptide from said plurality of database peptides;
(ii) identifying a modification site on said first peptide;
(iii) applying a modification to said modification site, producing a first modified peptide;
(iv) calculating a plurality of associated masses for said first modified peptide;
(v) selecting a first associated mass from said plurality of associated masses;
(vi) referencing a first record from said plurality of records, said first record corresponding to said first associated mass;
(vii) entering a first field into said first record, said first field comprising a first peptide index referencing said first peptide;
(viii) repeating steps (v)-(vii) for at least one other associated mass from said plurality of associated masses;
(ix) repeating steps (i)-(viii) for at least one other peptide from said plurality of database peptides.

12. The method of claim 11 wherein said identification step (ii) comprises the step of identifying a modification site selected from the group consisting of: a phosphorylation site, an oxidation site, and a substitution site.

13. The method of claim 12 wherein said phosphorylation site comprises an amino acid selected from the group consisting of: serine, threonine, and tyrosine.

14. The method of claim 12 wherein said oxidation site comprises an amino acid selected from the group consisting of: cysteine and methionine.

15. The method of claim 12 wherein said substitution site comprises an amino acid selected from the group consisting of: glutamine, glutamate, asparagine, and aspartate.
